(19) United States
(12) Patent Application Publication
(10) Pub. No.: US 2013/0242256 A1
(43) Pub. Date: Sep. 19, 2013

(54) METHOD AND APPARATUS FOR DETECTING ACCOMMODATION

(71) Applicants: Jean-Noel Fehr, Roanoke, VA (US); Urban Schnell, Roanoke, VA (US); Walter Doll, Roanoke, VA (US); Roland Michaely, Roanoke, VA (US); Jean-Christophe Roulet, Roanoke, VA (US); Amitava Gupta, Roanoke, VA (US); Ronald Blum, Roanoke, VA (US); Rudy Mazzocchi, Roanoke, VA (US)

(72) Inventors: Jean-Noel Fehr, Roanoke, VA (US); Urban Schnell, Roanoke, VA (US); Walter Doll, Roanoke, VA (US); Roland Michaely, Roanoke, VA (US); Jean-Christophe Roulet, Roanoke, VA (US); Amitava Gupta, Roanoke, VA (US); Ronald Blum, Roanoke, VA (US); Rudy Mazzocchi, Roanoke, VA (US)

(73) Assignee: Elenza, Inc., Roanoke, VA (US)

(21) Appl. No.: 13/822,569

(22) PCT Filed: Sep. 12, 2011
(86) PCT No.: PCT/US11/51198

§ 371 Date: Jun. 3, 2013

Related U.S. Application Data
(60) Provisional application No. 61/382,044, filed on Sep. 13, 2010.
(60) Provisional application No. 61/382,559, filed on Sep. 14, 2010.

(51) Int. Cl. A61B 3/11

(52) U.S. Cl. CPC A61B 3/11
USPC 351/205; 351/246

(57) ABSTRACT

A sensor system includes at least two sensors for distinguishing accommodative stimuli from changes in ambient lights levels and task-induced changes in the pupil diameter. When implanted, the first sensor is disposed completely within the pupil; even when fully constricted, the pupil does not occlude the first sensor, allowing the sensor to make precise measurements of ambient luminous flux levels. The pupil occludes part of the second sensor's active area(s) as the pupil dilates and constricts. As a result, the second sensor measures both ambient luminous flux and pupil diameter. A processor estimates the pupil diameter and determines whether it's changing in response to accommodative stimuli or other factors by comparing to predetermined values. The sensor system sends a signal to an optical component, which in turn can respond by changing optical power to focus for near vision upon detection of accommodative stimuli.

### CROSS-REFERENCE TO RELATED PATENT APPLICATIONS

This application claims priority from U.S. Provisional Application 61/382,044, filed Sep. 13, 2010, incorporated herein by reference in its entirety. This application also claims priority from U.S. Provisional Application 61/382,559, filed Sep. 14, 2010, incorporated herein by reference in its entirety.

### BACKGROUND

Accommodation is the process by which an eye focuses an image of an object less than six feet away. As we age, our accommodative amplitude decreases, and we lose the ability to focus on near objects. The loss of ability to focus on near objects is called presbyopia.

The natural lens can be replaced and/or supplemented with an artificial lens to enhance near vision. However, it has been problematic to create an artificial lens that provides suitable accommodation. Reading glasses and bifocals are inconvenient. Prior attempts to achieve an accommodative intraocular lens have also proved unsatisfactory. Such prior attempts have relied upon unreliable triggers that may give a false positive or false negative signal for accommodation. That is, they may signal for near vision when a near vision task is not present, or they may fail to signal for near vision when a near vision task is present.

There remains a need to satisfactorily detect the need for accommodation. By detecting a more specific accommodative stimulus, accommodation can more accurately be mimicked by an artificial optical component.

### SUMMARY

Embodiments of the present invention include a sensor system and corresponding method to detect a presence or absence of an accommodative stimulus in a human eye. Exemplary sensor systems include a first sensor configured to provide a first measurement indicative of an ambient light level and a second sensor configured to provide a second measurement indicative of a second light level that changes in response to a physiological change, such as a change in pupil diameter, triggered by the accommodative stimulus. The first and second measurements are used, e.g., by a processor, to determine the presence or absence of the accommodative stimulus based on the ambient light level and the physiological change.

In some embodiments, the first sensor is ring-shaped, i.e., it includes an annular active area that may have an inner diameter of about 0.9 mm to 1.2 mm and an outer diameter of about 1.1 mm to 1.3 mm. The second sensor may include an active area disposed along a diameter of the annular sensor, and an edge of this active area and an outer diameter of the annular sensor may further define a gap having a length of about 250-600 μm. The active area can have a width of about 30 μm to about 300 μm. The first sensor may also include another active area disposed along the diameter of the annular sensor. At least one of the first and second sensors can include or be formed of a photovoltaic cell.

Further embodiments may include a processor that is operably coupled to the first and second sensors and configured to determine the presence or absence of the accommodative stimulus based on the first and second measurements. For example, the processor may be configured to determine the presence or absence of the accommodative stimulus by comparing the first measurement and the second measurement to predetermined values representing the second measurement as a function of the first measurement in the presence of the accommodative stimulus, e.g., by fitting a curve or looking up values stored in a memory. The predetermined values may be specific to the patient and can be chosen based on the patient's age, psychological stress, and/or physiological health. The processor may determine a status of the human eye and/or an environment of the human eye.

Alternatively, the processor may determine the presence or absence of the accommodative stimulus by (i) computing a ratio of the first measurement to the second measurement, and (ii) estimating the pupil diameter based on the ratio of the first measurement to the second measurement. In embodiments where the second sensor includes first and second active areas (e.g., one opposite sides of the first sensor), and the processor may determine the presence or absence of the physiological change by computing a difference in photocurrent generated by the first and second active areas.

In yet further embodiments, the sensor system includes a memory that is operably coupled to the first and second sensors and configured to store representations of the first and second measurements acquired over a predetermined interval, e.g., of about 0.25 seconds to about 0.50 seconds. The processor may (i) compute running averages of the first and second measurements acquired over the predetermined interval, and (ii) compare the running averages to predetermined values representing the presence or absence of the accommodative stimulus. The memory may be a nonvolatile storage medium such as an electrically erasable programmable read-only memory (EEPROM).

Still further embodiments of the sensor system comprise an electro-active element that is operably coupled to the processor and configured to provide a change in effective optical power and/or depth of field in response to a signal from the processor. The processor may also place the electro-active cell in an inactive mode when at least one of the first and second measurements indicates that the ambient light level corresponds to an illuminance of less than about 5 lux. In addition, the processor may place the electro-active cell in an active mode when at least one of the first and second measurements indicates that the ambient light level corresponds to an illuminance of greater than about 5 lux.

Yet other embodiments of the present invention include a sensor system that comprises a first photosensor, a second photosensor, and a processor. When the sensor system is implanted in a human eye, the a first and second photosensors are positioned at first and second distances, respectively, from the pupil. In some instances, the first distance is about 0.45 mm to about 0.60 mm and the second distance is about 0.80 mm to about 1.25 mm. The processor detects the accommodative stimulus, e.g., based on a ratio of the outputs from the first and second photosensors. The sensor system may be calibrated for an individual patient.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The accompanying drawings, which are incorporated in and constitute a part of this specification, illustrate embodiments of the disclosed technology and together with the description serve to explain principles of the disclosed technology.

FIG. 1 is a plot of pupil diameter versus brightness for different ages and distances D0 from the eye to an object of regard.

FIG. 2 is a plot of accommodation-induced change in pupil diameter versus objects at different distance for different ages and ambient illumination levels.

FIGS. 3A and 3B are schematic diagrams of an implanted intra-ocular optic (IOO) with a variable-diameter dynamic aperture in absorbing and transmitting states, respectively.

FIG. 4 is a schematic diagram of a dynamic IOO with an inventive multiple sensor system that detects both an ambient light level and a physiological response with an annular first sensor and a second sensor with a pair of linear active areas disposed on either side of the first sensor.

FIG. 5 is a schematic diagram illustrating how the pupil affects the amount of light detected by first and second sensors in an implanted dynamic IOO according to embodiments of the present invention.

FIG. 6 is schematic diagram that illustrates how the illuminated portions of the second sensor's active areas change as function of pupil diameter.

FIG. 7 illustrates how the ratio of illuminated sensor areas versus brightness (left) and the digital value (gray scale) versus brightness for the first sensor (middle) can be used to determine the digital value (gray scale) versus brightness for the second sensor (right) in response to accommodative stimuli.

FIGS. 8A and 8B illustrate the effects of changing the width of the second sensor's active areas and the gaps between the second sensor's active areas and the annular first sensor in of FIG. 6 for on accommodative stimuli associate with objects at different ranges of regard.

FIG. 9 is a plot of error in illuminated area (measured in percent) versus offset distance for the an illustrative multiple sensor system.

FIG. 10 is a plot of gray level (digital output) for the second sensor versus gray level (digital output) for the first sensor in an illustrative multiple sensor system for different values of vertical and horizontal offsets of the multiple sensor system with respect to the pupil.

FIGS. 11A-11F illustrate error in illuminated area (measured in percent) versus offset distance for the multiple sensor systems with different numbers and arrangements of active areas.

FIGS. 12A-12F illustrate error in illuminated area (measured in percent) versus offset distance for the multiple sensor systems with different numbers and arrangements of active areas used with an acircular pupil.

FIGS. 13A-13D illustrate construction and use of a look-up table (LUT) to detect an accommodative stimulus with digital outputs D₁ and D2 from the first and second sensors of FIG. 4.

FIG. 14 illustrates a sensor system with a processor that determines the presence or absence of an accommodative stimulus based on comparisons of measurements from first and second sensors to entries in the LUT shown in FIG. 13C and actuates a dynamic aperture if an accommodative stimulus is present.

FIG. 15 is a plot that shows a bilinear fit to the gray scale plot at right in FIG. 7

FIG. 16 illustrates a sensor system with a processor that determines the presence or absence of an accommodative stimulus based on fits of measurements from first and second sensors to the bilinear fit shown in FIG. 15 and actuates a dynamic aperture if an accommodative stimulus is present.

FIG. 17 is a diagram that illustrates iteration through the loop implemented by the system of FIG. 16.

### DETAILED DESCRIPTION

### Pupillary Responses Due to Accommodative Stimuli, Cognitive Tasks, and Ambient Light

Inventive sensor systems include two or more sensors configured to make measurements of an ambient light level and a physiological change, such as a change in pupil diameter, that occurs in response to an accommodative stimulus. By using both pupil constriction and ambient light as criteria for the accommodative trigger, the sensor system more accurately detects the need for accommodation. For example, pupil constriction occurs with accommodation, but it also occurs with increased ambient light. Thus, relying on pupil constriction as the sole accommodative trigger may generate false positive accommodation signals. By adding an ambient light criterion, the sensor system can detect circumstances where the pupil is dilated but the ambient light is not high, thus increasing accuracy. Similarly, relying on intermediate ambient light as the sole accommodative trigger may generate false positive accommodation signals, such as when viewing an object across a room.

Including two criteria, e.g., pupil constriction and ambient light, also improves accuracy under atypical vision tasks, such as reading a menu in a darkened restaurant. Other atypical vision tasks, such as reading a book at the beach, may be accomplished by simply using sunglasses to reduce the amount of ambient light near the corneal surface to below the ambient light upper limit and/or within the range of intermediate ambient light.

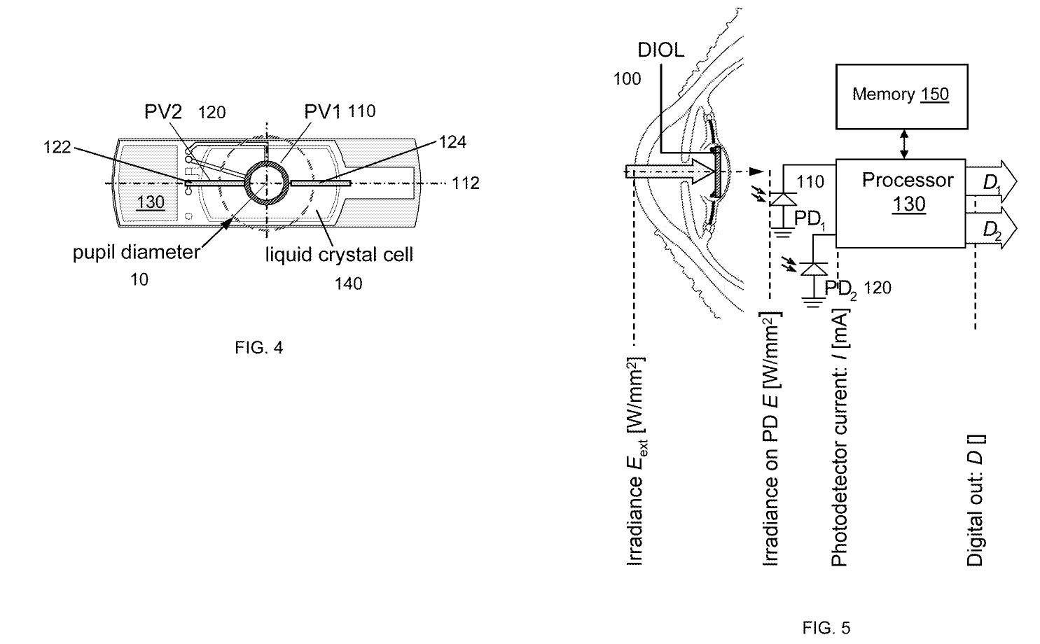

In healthy humans, accommodation induces a combination of physiological responses known as the accommodative triad: convergence, pupil constriction, and change of power of the crystalline lens. Of these physiological responses, convergence may be monitored using a micro-accelerometer, while changes in pupil size may be monitored by measuring illuminance at the plane of an intra-ocular lens (IOL) (e.g., using the second sensor 120 shown in FIGS. 4-6). Changes in pupil diameter, or pupillary responses, can be divided into four categories: (1) changes caused by cognitive processes and behavioral activities (task-induced changes); (2) changes caused by accommodative stimuli; (3) changes caused by blinking (momentary constriction flowed by a return to the resting value); and (4) changes caused by application of drugs. Distinguishing between task-induced changes and changes caused by accommodative stimuli enables proper compensation for loss of accommodation.

Task-induced pupillary responses occur in response to stimuli that include, but are not limited to cognitive stimuli such as fatigue, pain (which may result in pupil diameter changes of about 0.25-0.37 mm), and sexual stimuli; psychological triggers such as sadness, nonverbal communication; and mental activities such as cognitive tasks, short term memory, language processing; and so on. Task-induced pupillary responses show lag from the onset of the task stimulus and an inverse power law dependence of the pupil diameter on time measured from the task stimulus as shown, e.g., in E. Granholm, et al., “Pupillary responses index cognitive resource limitations,” Psycophysiology, 33, 457-461 (1996), which is incorporated herein by reference in its entirety.

In contrast, the pupillary response to an accommodative stimulus shows no lag in time and is exponential in character, as shown, for example, in S. Kasthurirangan and A. Glasser, “Age related changes in the characteristics of the near pupil response,” Vision Research, 46, 1393-2003 (2006). Studies show a close correlation between the accommodative stimulus and changes in pupil size following an exponential law. The magnitude of the pupillary response, as measured by the slope of the change in pupillary diameter versus accommodative stimulus (e.g., in mm/Diopter) increases with age.

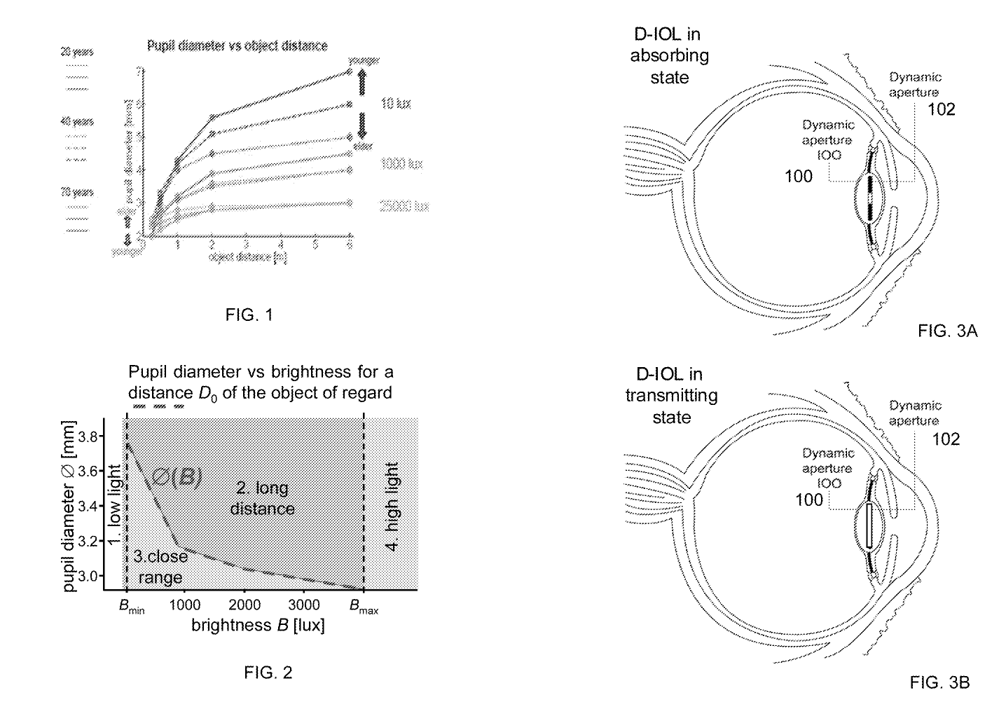

The pupil size also varies with ambient illumination level as shown in FIG. 1, which is a plot of pupil diameter versus brightness for an eye fixed on an object at a distance D₀. Each brightness/pupil diameter corresponds to a particular range to an object. The range increases with pupil diameter for a given ambient light level. It also increases with ambient light level for a given pupil diameter. A dashed line indicates the boundary between objects at long distances (e.g., distances greater than about 900 mm) and objects at close ranges (e.g., distances of about 900 mm or less). In some cases, an implantable ophthalmic device may provide static optical power for viewing objects at long distances and a variable optical power and/or depth of field for viewing objects at close ranges, e.g., by varying the diameter of a dynamic aperture and/or actuating an electro-active element. At light levels below a minimum brightness Bₘᵢₙ (e.g., about 5 lux), the pupil diameter does not change as a function of ambient light level because the eye cannot detect any light (and may actually be closed). Similarly, the pupil diameter does not change for light levels above a maximum brightness Bₘₐₓ (e.g., more than about 25,000 lux).

Precise measurements of ambient light levels are especially useful for detecting accommodative stimuli when performing near vision tasks, such as reading, under intermediate ambient light, e.g., indoor lighting conditions. The variability of indoor lighting conditions is much less than the variability of outdoor lighting conditions. For example, the amount of light outdoors can vary by as much as seven orders of magnitude from full sunlight to nighttime darkness. In contrast, the amount of light typical indoors varies by as little as one-half order of magnitude.

Other factors, including age, also affect the pupil diameter, as shown in FIG. 2, which is a plot of pupil diameter versus object distance for 20-year-old, 40-year-old, and 70-year-old subjects at ambient light levels of 10 lux, 1000 lux, and 25,000 lux. The pupil size varies by 3-4 mm, depending on age, as a function of ambient illumination. It also varies by 1-6 mm, depending on ambient illumination level, as a function of object distance. FIG. 1 also shows that the accommodative response of pupil size diminishes as the ambient illumination level increases. If ambient illumination level exceeds an upper threshold, the degree of pupil dilation caused by the accommodative response by the pupil may be too to determine the presence/absence of an accommodative stimulus with a high level of confidence.

Sensor Systems to Detect Pupillary Responses Associated with Accommodative Stimuli

FIGS. 3A and 3B show an implantable ophthalmic device 100 implanted in a human or other animal eye to compensate for the loss of or degradation in accommodative response (defined below) due to presbyopia and/or other ophthalmic conditions. The implantable ophthalmic device 100 includes a dynamic aperture 102 that closes (FIG. 3A) and opens (FIG. 3B) to increase the effective optical power and/or depth of field of the eye as described in U.S. Pat. No. 7,926,940 to Blum et al., which is incorporated herein by reference in its entirety. In certain embodiments, the dynamic aperture 102 is implemented using an annular electro-active element that reflects and/or absorbs incident light in response to measurements by a multiple sensor system (not shown) that detects both ambient light levels and physiological changes, such as changes in pupil diameter, caused by an accommodative stimulus.

FIG. 4 shows the active portion of the implantable ophthalmic device 100 with a first sensor 110 and a second sensor 120 that includes a left active area 122 and a right active area 124 disposed along a diameter 112 of the first sensor 110. The first sensor 110 has an annular active area with an inner diameter of about 0.9-1.2 mm, an outer diameter of about 1.1-1.3 mm, and a fill factor (ratio of active area to exposed sensor area) of about 100%. (By comparison, the pupil diameter 10 of a healthy adult human ranges from about 2.0-6.0 mm depending on ambient light levels, range of regard, and other factors.) A gap of about 250-600 μm extends between the outer diameter of the first sensor 110 and the inner edges of the left and right active areas 122, 124, which may be up to several millimeters long (e.g., 2 mm, 3 mm, 4 mm, 5 mm, 6 mm, 7 mm, or any length between any two of these values) and about 30-300 μm wide. Those of skill in the art will readily appreciate that other sensor shapes and sizes fall within the scope of the present disclosure.

When the implantable ophthalmic device 100 is implanted properly, the first sensor 110 is concentric with and completely within the diameter 10 of the pupil and the active areas 122, 124 of the second sensor 120 are symmetric about the center of pupil. The first sensor 110 is typically positioned closer to the center of the pupil than the second sensor 120, but in general, the terms “first” and “second” are used to merely as a naming convention rather than to define any particular characteristics or orientation. For example, the first sensor 110 may be implanted at a distance of about 0.45-0.55 mm from the center of the pupil, and the second sensor 120 may be implanted at a distance of about 0.80-1.25 mm from the center of the pupil.

In operation, the first sensor 110 and second sensor 120 detect the brightness, or illuminance, of incident light 12 as shown in FIG. 5. As understood by those of skill in the art, the illuminance is the total luminous flux per unit area (usually expressed in lux or lumens/m²). The illuminance in lux can be converted to irradiance, or power per unit area (usually expressed in W/m²), with knowledge of a wavelength-dependent conversion factor representing the eye's luminosity function. The first and second sensors 110, 120 detect irradiance over a given active area (i.e., power) and produce first and second photocurrents I1, I2, respectively, whose amplitudes are proportional to the illuminance of the incident light 12. In preferred embodiments, the first and second sensors 110, 120 have similar responsivities (quantum efficiencies), sensitivities, and dynamic ranges. A processor 130 in electrical communication with the sensors 110, 120 converts the photocurrents from the first and second sensors 110, 120 to first and second digital outputs D₁, D2 that correspond to gray levels and/or diameters of the dynamic aperture 102. As explained in greater detail below, the processor 130 may determine the first and second digital outputs D₁, D2 by comparing the measurements represented by the photocurrents to measurements stored in a memory 150. The processor 130 may also store indications of one or more photocurrent values in the memory 150 and compute running averages of the photocurrents based on the values stored in the memory 150.

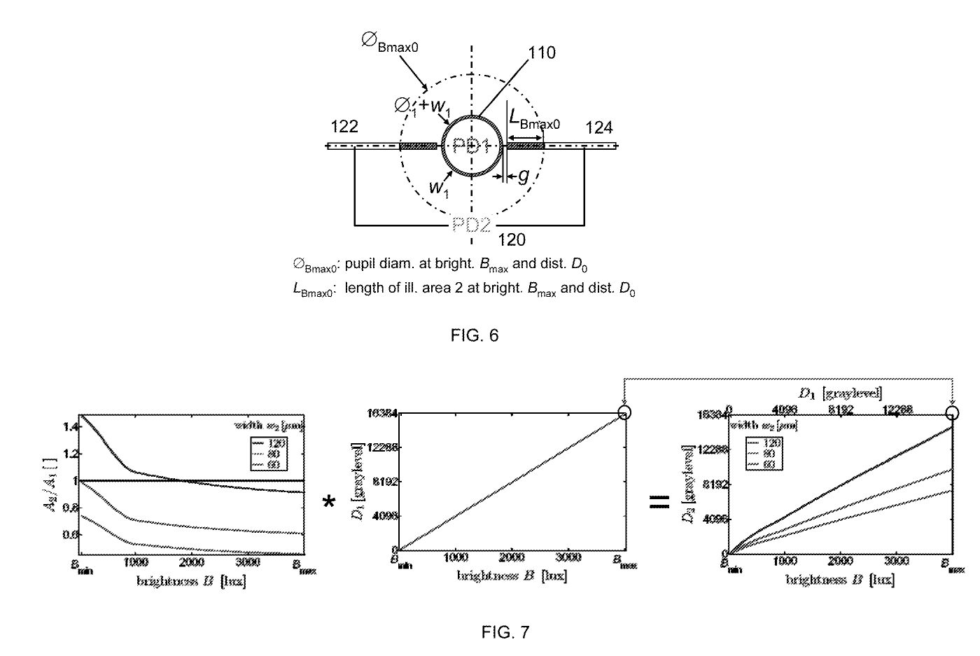

FIG. 6 illustrates how the change in pupil diameter 10 affects the amplitudes of the photocurrents generated by first and second sensors 110, 120. The outer diameter of the first sensor 110 is smaller than the minimum pupil diameter 10, so the entire active area of the first sensor 110 is always illuminated (provided that the eyelid is open). As a result, the amplitude of the photocurrent I1 emitted by the first sensor 110 varies with the ambient light level, but not the pupil diameter 10. Conversely, the pupil occludes or obscures portions of the active areas 122, 124 as it opens and closes in response to changes in ambient light levels and/or accommodative stimuli, which causes the amplitude of the photocurrent I2 emitted by the second sensor 120 to vary with both the ambient light level and the pupil diameter 10.

Because the first and second sensors 110, 120 are positioned at different distances from the center of the pupil, the differential between the photocurrents I1, I2 generated by the first and second sensors 110, 120 can be used to determine the degree of pupil constriction. For example, when the first sensor 110 is positioned closer to the center of the pupil than the second sensor 120, the second sensor 120 is eclipsed by the constricting iris before or to a greater degree than the first sensor 110. When the pupil is dilated, both sensors 110, 120 may be exposed to ambient light. When the pupil is partially constricted, the second sensor 120 may be eclipsed by the iris, while the first sensor 110 remains exposed. When the pupil is further constricted, both photosensors may be eclipsed by the iris, but the second sensor 120 may be eclipsed to a greater degree than the first sensor 110. As a sensor 110 becomes more eclipsed, the amount of light detected at that ocular position will decrease.

More specifically, the amplitude of the photocurrent from the first sensor 110 can be expressed as I₁=A₁·∫R₁(λ)·S₁(λ) dλ, where A₁ is the active area of the first sensor 110, R(λ) is the irradiance on the active area of the first sensor 110, S(λ) is the spectral sensitivity of the first sensor 110, and λ is the wavelength. The amplitude of the photocurrent from the second sensor 120 is given by I₂=A₂(Ø)·∫R₂(l)·S₂(l) dl, where Ø is the pupil diameter. The photocurrent from the first sensor 110 can be approximated as I₁∞A₁·B, where B is the brightness (illuminance). The photocurrent from the second sensor 120 can likewise be approximated as I₂∞A₂(Ø)·B.

The processor 130 uses the measurements of ambient light level and pupil diameter represented by the first and second photocurrents I1, I2 from the first and second sensors 110, 120 to determine whether or not an accommodative stimulus is present. For example, the processor 130 may determine that an accommodative stimulus is present based on a pupil diameter 10 estimated from a ratio of the signals from the first and second sensors 110, 120 and measured brightness (represented by the signal from the first sensor 110) as shown in FIG. 7. The processor 130 takes the ratio of illuminated sensor areas A2/A1, which is proportional to the ratio of photocurrents. Ratios that lie along the curves plotted at left in FIG. 7 for different widths w2 of the second sensor's active areas 122 and 124 are associated with accommodative stimuli. Multiplying the illuminated sensor area ratio with the first sensor's gray level D₁ response versus brightness (plotted in the center of FIG. 7) yields the second sensor's gray level D2 response, plotted at right in FIG. 7 versus brightness (bottom axis) and the first sensor's gray level D₁ (top axis) in the presence of an accommodative stimulus.

If the processor 130 senses an accommodative stimulus, it actuates an electro-active element—e.g., a liquid crystal cell 140 as shown in FIG. 4—that provides the variable diameter aperture 102 described above. The liquid crystal cell 140 may be pixelated or otherwise segment to provide a continuous or nearly continuous range of diameters and/or levels transmission in response to commands from the processor 130, which may be an application-specific integrated circuit (ASIC) as described in PCT/US2011/040896 to Fehr et al., which is incorporated herein by reference in its entirety. The implantable ophthalmic device 100 may also include a rechargeable battery or other power supply for powering the processor 130 and the liquid crystal cell 140 as well as an inductive coil or other antenna for remotely recharging the battery and/or communicating with the processor 130 as described in PCT/US2011/050533 to Fehr et al., which is also incorporated herein by reference in its entirety.

The first and second sensors 110, 120 may be implemented as photovoltaic cells, photodiodes, photosensors, or any other suitable device that produces a change in voltage, current, resistance, and/or other measurable quantity when illuminated with visible light, i.e., light within a wavelength range of about 400-700 nm. As understood by those of skill in the art, a photovoltaic cell includes a junction defined by an n-doped semiconductor layer adjacent to a p-doped semiconductor layer. Suitable semiconductor materials include, but are not limited to gallium arsenide, silicon, cadmium telluride, and/or copper indium gallium selenide/sulfide. The doping creates a built-in electric field that sweeps electron-hole pairs created by absorption of incident photons such that the voltage across the junction increases with increasing photon flux.

The implantable ophthalmic device 100 may also include one or more lens elements that provide static optical power in addition to the dynamic effective optical power and/or depth of field provided by the dynamic aperture 102. In cases where the implantable ophthalmic device is an intraocular lens (IOL), the IOL may have at least one static optical power provided by a curved surface and/or a graded index profile. For example, the IOL may include spherical optical element and/or an aspheric optical element as described in PCT/US2011/038597 to Blum et al., which is incorporated herein by reference in its entirety. Alternatively, the implantable ophthalmic device may be an intraocular optic (IOO), which has little to no optical power, but also includes a dynamic aperture that provides an increased depth of field. In some illustrative devices with dynamic apertures, opening and closing the aperture serves to provide a continuous range of focus between the fixed or static corrective powers of the ophthalmic lens.

Inventive sensor systems can be embedded in or affixed to an IOL, IOO, corneal inlay, corneal onlay, or other implantable ophthalmic device. Implantable ophthalmic devices, such as the device 100 of FIG. 1, may be inserted or implanted in the anterior chamber or posterior chamber of the eye, into the capsular sac, or the stroma of the cornea (similar to a corneal inlay), or into the epithelial layer of the cornea (similar to a corneal onlay), or within any anatomical structure of the eye. When implanted, the first and second sensors 110, 120 are positioned on substantially the same coronal plane. For example, when the sensors are integral with an IOL, the sensors are positioned on the plane of the IOL.

As described above, two or more sensor active areas may be implanted or otherwise positioned along the same diameter (so that they form a single line through the center of the pupil) or along different radii at different distances from the center of the pupil (or the center point of the device ex vivo, which aligns with the center of the pupil in vivo). The distance between a sensor and the center is designated “d.” The first sensor is a first distance, d₁, from center. The second sensor is a second distance, d₂, from center, and so on. In one embodiment, each distance d is about 0 mm to about 7 mm. In one embodiment, each distance d is independently selected from: about 0.1 mm, about 0.2 mm, about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, and about 7 mm. In one embodiment, the sensors are positioned relative to the edge of the iris under intermediate ambient light. For example, the first sensor 110 can be positioned closer to center than the iris edge such that the active area of the first sensor 110 is exposed, and the second sensor 120 can be positioned farther from center than the iris edge such that the second sensor 120 is eclipsed by the iris as explained below.

The sensor system may be calibrated for an individual patient. Pupil size and constriction response vary among individuals and may even vary between eyes of the same individual. The sensor system may be calibrated by one or more of: altering the position of one or more sensors, modifying the accommodative trigger function, modifying the sensitivity of the system (e.g., the pupil constriction lower limit and/or the ambient light upper limit).

### Sensor Geometry and Alignment

Referring again to FIG. 6, the geometry of the first and second sensors 110, 120 is chosen to exploit the sensors' entire dynamic range(s) and to provide robustness to offsets of up to about of ±0.1 mm with respect to the to optical axis. In preferred embodiments, the width w₁ of the first sensor 110 provides a maximum photocurrent for the largest expected brightness and minimum photocurrent for the smallest expected brightness. For example, the width w₁ may be chosen such that the maximum and minimum photocurrents correspond to digital values (gray levels) D₁ of 16,000 and 160, respectively.

As discussed above, the illuminated area of the second sensor 120 depends on the pupil diameter Ø, the gap g, the length L of the illuminated area, and the inner diameter of the first sensor Ø₁: A₂=2·w₂·L=w₂·[Ø−Ø₁−w₁−2·g]. The pupil diameter for the maximum brightness Bₘₐₓ varies slightly with the distance of the object of regard: for a distance of regard D₀=0.90 m, the pupil diameter may be about 2.9 mm at the maximum brightness, Bₘₐₓ; whereas the pupil diameter may be about 3.5 mm for a distance of regard D₀=5.00 m at the same brightness. Choosing the width w₂ₘₐₓ to be about equal to A₁/(2·LBₘₐₓ₀) sets the maximum and minimum photocurrents generated by the second sensor 120 to be about to equal to those generated by the first sensor 110. A second sensor 120 with a width w₂<w₂ₘₐₓ can measure pupil diameters that are slightly bigger than the diameter at the maximum brightness threshold for a given distance of regard. Choosing a smaller width w₂ implies: lower dynamic range; less robustness for pupil diameter estimation due to the decreased intersection area with the iris; greater robustness for measurements at high brightness, which allows for measurements of bigger pupil diameters; and less transmission loss through the sensor system.

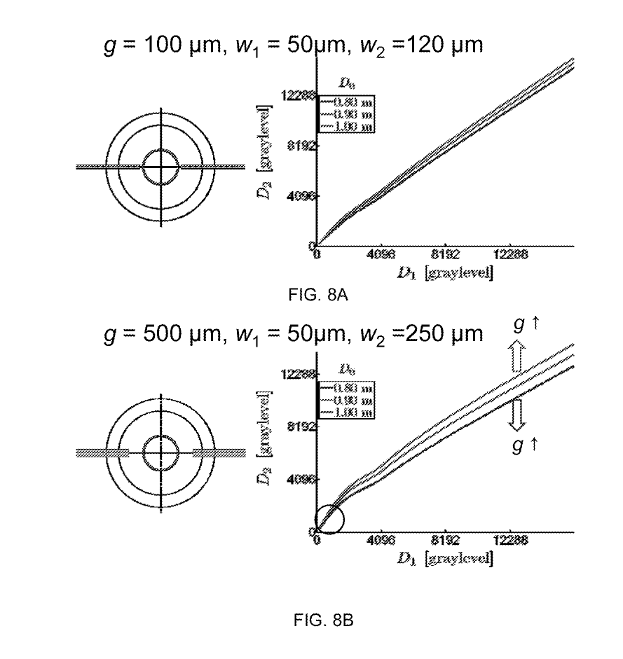

FIGS. 8A and 8B shows that the gap g between the first and second sensors 110, 120 also affects the performance of the sensor system. FIG. 8A is a plot of gray level D2 versus gray level D₁ for a gap of 100 microns, a first sensor width w1 of 50 microns, and a second sensor width w2 of 120 microns for distances of regard of 800 mm, 900 mm, and 1 m. FIG. 8B is a plot of gray level D2 versus gray level D₁ for a gap of 500 microns, a first sensor width w1 of 50 microns, and a second sensor width w2 of 250 microns for distances of regard of 800 mm, 900 mm, and 1 m. Taken together, FIGS. 8A and 8B show that smaller gaps lead to smaller variations in the value of gray scale D2 for a given range of distances of regard.

A lower gap distance implies a large offset in photocurrent (and digital values) for the second sensor 120 and a smaller variation in photocurrent (and digital values) for objects at different distances of regard. Choosing a bigger gap g implies a bigger tolerance in the values of D₂ for the discrimination of the vision range in the bigger brightness range at the expense of larger widths w2 for equivalent dynamic range. In addition, if the gap g gets small, the start photocurrent can become lower than the noise level.

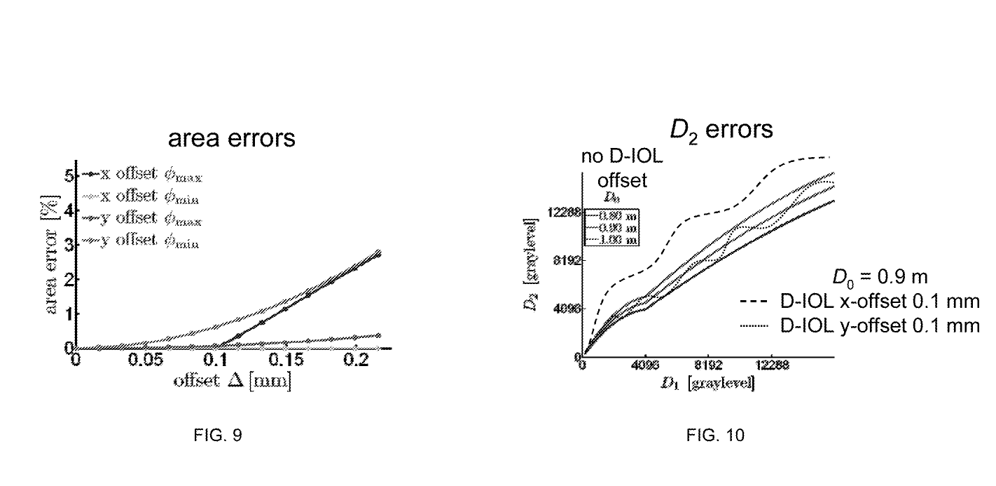

Misalignment of the sensors 110, 120 with respect to the pupil also affects the performance of the sensor system. As shown in FIG. 9, which is a plot of error in illuminated area for different horizontal (x) and vertical (y) offsets, the error due to maximum horizontal offset asymptotically approaches the error due to the maximum vertical offset. The gap geometry determines the y-intercept of the error due to maximum horizontal offset. The offset errors also introduce ripples and bias error into the scaling of the second sensor gray scale D2 with first sensor gray scale D₁ as shown in FIG. 10.

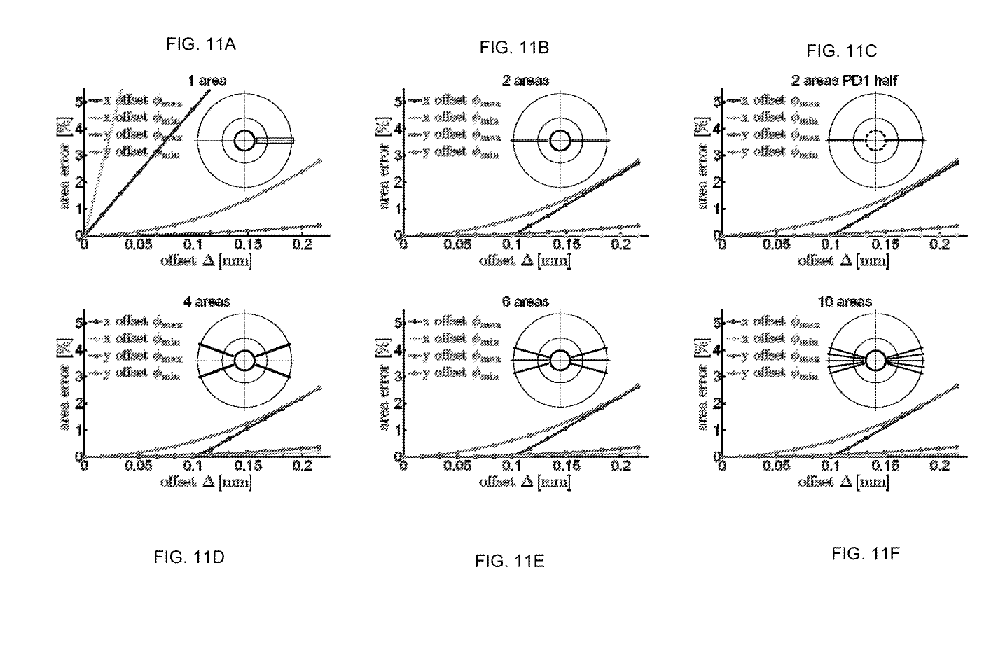

FIGS. 11A-11F show simulated percent errors in illuminated active area of the second sensor 120 versus offset for alternative arrangements of sensor active areas for pupil diameters of 2.70 mm and 5.65 mm. In FIG. 11A, the second sensor 120 includes only one active area with a width of w2=300 μm disposed along a radius of the first sensor 110. FIGS. 11B and 11C show errors for a second sensor with two active areas (widths w2=150 μm and w2=80 μm, respectively) and first sensors with 100% and 50% fill factors, respectively. FIGS. 11D, 11E, and 11F show plots for four active areas (w2=80 μm), six areas (w2=50 μm), and ten areas (w2=30 μm), respectively. The symmetric active area arrangements shown in FIGS. 11B-11F have robust performance with respect to offset error, whereas as the asymmetric active area arrangement shown in FIG. 11A is relatively intolerant to offset error and more sensitive to the direction of error. The area errors are bigger for smaller pupil diameters (low light) and if the pupil diameter is greater than the biggest measurable pupil diameter. In addition, the area error is not symmetrical.

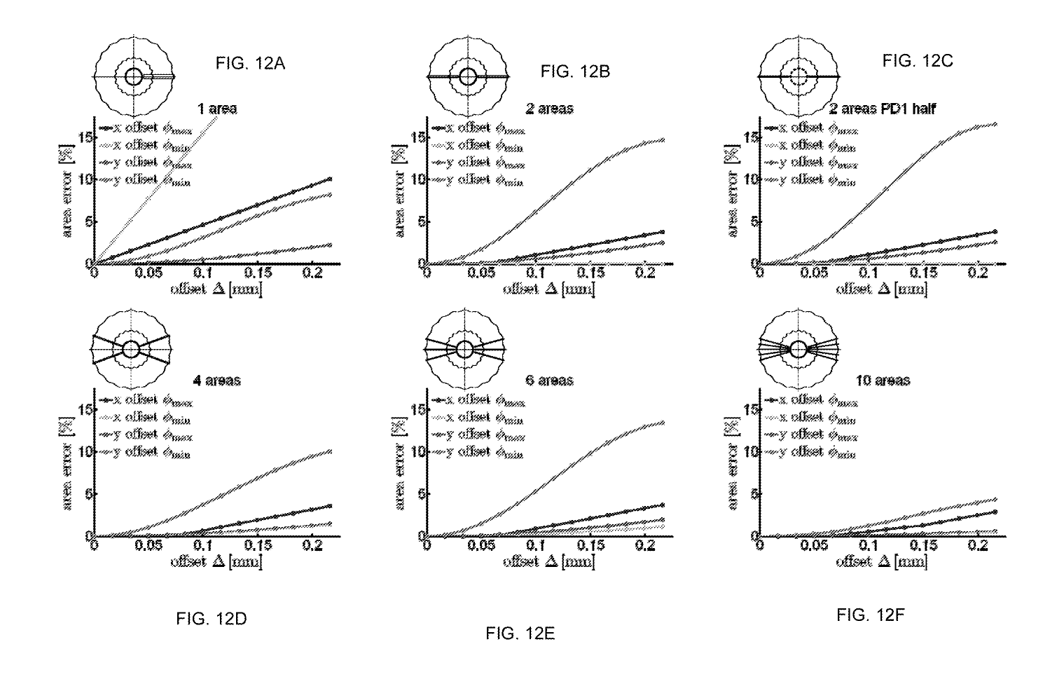

FIGS. 12A-12F show simulated errors in illuminated active area of the second sensor 120 versus offset results for an acircular pupil for the same sensor arrangements illustrated in FIGS. 11A-11F. The pupil was simulated by superposing sinusoid with a frequency of 20 oscillations per unit length and an amplitude of 0.1 mm onto a circular iris opened to diameters of 2.70 mm and 5.65 mm. The simulations show that area errors are bigger for small pupil diameters (lower light levels) for acircular pupils, that arrangements with one horizontal active area for the second sensor 120 are not robust to horizontal offsets, and that all other sensor architectures show very similar results. Increasing the number of active areas (e.g., to ten or more, as shown in FIG. 12F) reduces the percent error.

Detecting Accommodative Stimuli with Multiple Sensor Systems

In preferred embodiments, the processor 130 (FIG. 4) actuates the dynamic aperture 102 (FIG. 1) to improve near vision (without compromising intermediate and far vision) in response to measurements from the first and second sensors 110, 120 based on data and/or instructions stored in the memory 150. When the sensors 110, 120 indicate that ambient light levels have fallen below a minimum threshold (e.g., about 40 lux), the processor 130 (at least partially) opens the aperture 102 to minimize light reduction in low-light conditions. The processor 130 also (at least partially) opens the dynamic aperture 102 when the ambient light is within a tolerated range and the object of regard is at a medium or long distance (e.g., greater than about 900 mm). The processor 130 (at least partially) closes the dynamic aperture 102 when the object of regard is closer than about 900 mm and/or the ambient light level exceeds an upper threshold (e.g., about 4000 lux).

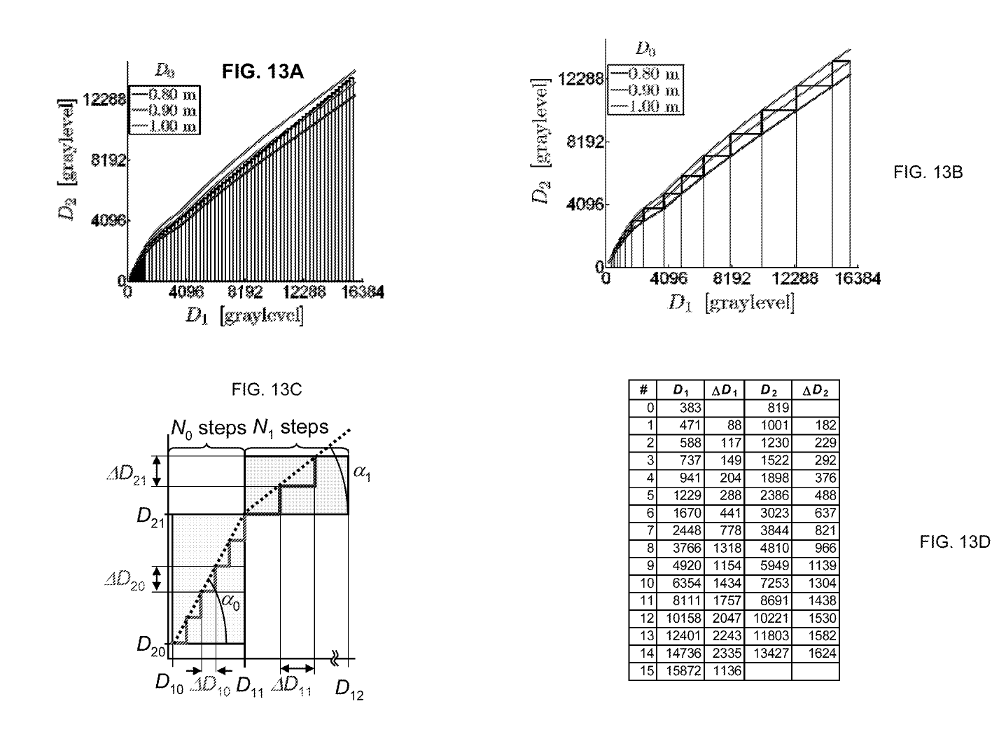

FIGS. 13A-13D illustrate construction of a look-up table (LUT) that can be stored in the memory 150 and used to determine the presence or absence of an accommodative stimulus based on the digital outputs D₁ and D2 from the first and second sensors 110 and 120, respectively. FIGS. 13A and 13B are plots of the second digital output D2 versus the first digital output D₁ in response to a change in pupil diameter associated with objects at distances of 0.80 m, 0.90 m, and 1.00 m. The curves are bilinear—the second digital output D2 changes with respect to the first digital output D₁ at a faster rate (e.g., a slope of about two) over a segment from the origin to about D₁=2048, after which the slop is roughly one. In FIG. 13A, the segments are fit to a single curve; whereas, in FIG. 13B, the segments sampled between curves representing objects separated by a distance of about 0.2 m.

FIG. 13C illustrates the evolution in the digital outputs in the presence of an accommodative stimulus with a staircase sampling (solid line) obtained by sampling a bilinear envelope (dotted line) representing a fit to one of the curves plotted in FIG. 13A and/or a fit bounded by two curves as shown in FIG. 13B. The first and second bilinear regions of the staircase sampling are divided into N0 and N1 steps, respectively, and have slopes of a0 and al, respectively. Each corner on the staircase represents a pair of measurements with coordinates (D₁ᵢ, D₂ᵢ), where i is an index representing the order in which the measurements were taken. In this example, D₁₀ represents the digital output of the first sensor 110 at t=0, D₁₁ represents the digital output of the first sensor 110 at t=1, and so on. The measurements are spaced apart by distances ΔDxi=Dxi−Dxi−1 (e.g., ΔD₁₁=D₁₁−D₁₀).

The data represented in FIG. 13C, which may be different for different people, may be used to compile a look-up table (LUT) like the one shown in FIG. 13D. Each row of the LUT includes a pair of sampled coordinates (D₁, D2) as well as the local run and rise (ΔD₁, ΔD2) of the bilinear curve near the sampled coordinates. In some examples, an ophthalmologist or technician may construct the LUT (possibly using a computer) based on an evaluation of the patient.

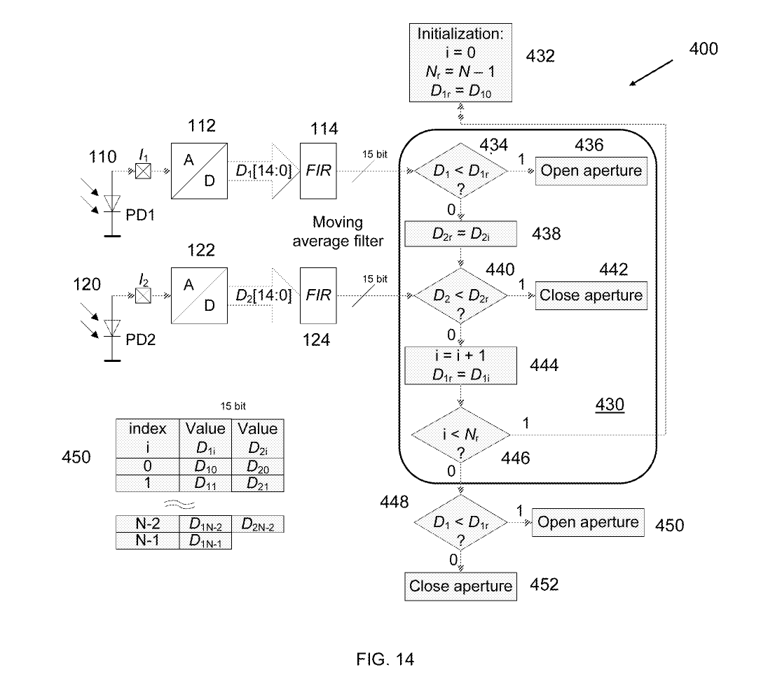

FIG. 14 illustrates how a system 400 determines the presence or absence of an accommodative trigger and actuates the dynamic aperture 102 (e.g., as provided by an electro-active aperture) using a LUT 450 stored in memory 150 (FIG. 3). The first and second sensors 110, 120 generate photocurrents I1, I2 which are digitized by analog-to-digital converters 112, 122 to provide the digital outputs D₁, D2 about once every 0.25-0.50 seconds. Finite impulse response (FIR) filters 114, 124 take running averages of the digital outputs and store the running averages in a data registry (memory 150, shown in FIG. 3) that also stores the most recent 50-100 digital values, moving averages, and/or estimated pupil diameters.

A processor 430 compares the moving averages to thresholds using the following loop. The subscripts i and r indicate values associated with the current loop iteration and previous loop iteration, respectively. First, the processor 530 initializes the loop index i, the number of iterations Nr, and the value of D₁ᵣ to D₁₀, which represent the minimum brightness for active mode (e.g., about 5 lux), in step 432. Depending on whether the initial values for D1 and D2 are in the first or second region of the bilinear curve, the number of iterations Nr is either N0 or N1 (shown in FIG. 13C).

Once initialization is over, the processor 430 compares D₁, which represents the ambient light level, to D₁₀, which represents the minimum ambient light level in step 434; if D₁<D₁₀, the processor 430 opens the dynamic aperture 102 in step 436. If not, the processor 430 updates D2r, which represents the preceding value of D2, in step 438. It then compares the current D2, which represent the pupil diameter, to the preceding value D2r in step 440. If the pupil diameter has gotten smaller, the processor 430 closes the aperture to change the effective optical power and/or depth of field in step 442. The processor 430 increments i and D₁ᵣ in step 444, checks i against Nr in step 446 and either re-initializes the loop or checks the ambient light level against the minimum threshold and opens or closes the aperture accordingly (steps 448, 450, and 452).

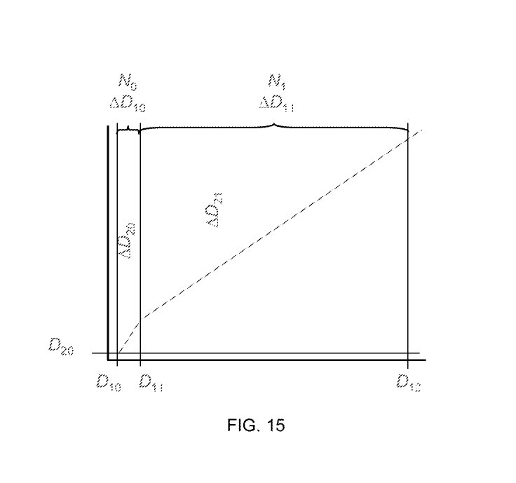

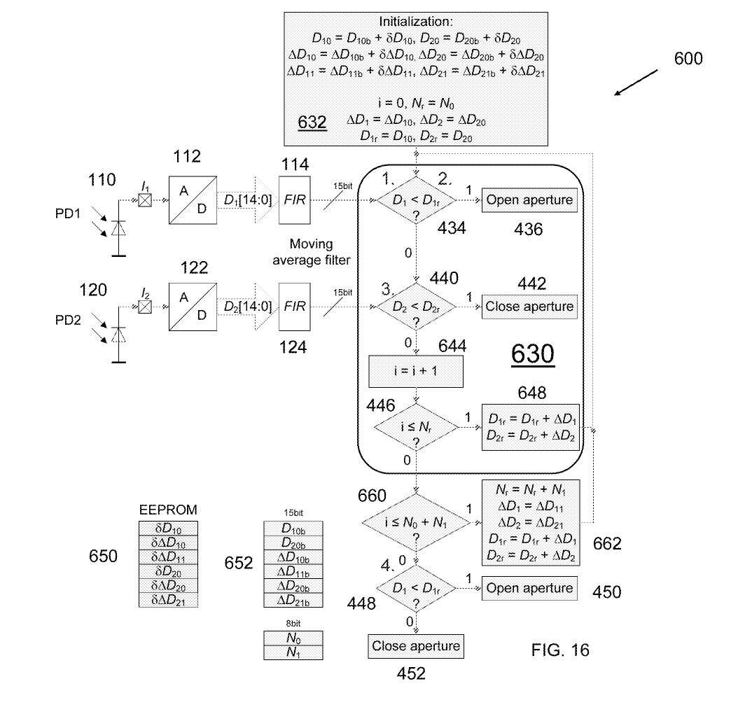

FIG. 16 shows an alternative system 600 that fits the digital outputs D₁, D2, to the bilinear curve shown in FIG. 15 using values for slopes δDₓᵢ and derivates of slopes δΔDₓᵢ for different regions of the bilinear curve. As above, the system 600 includes a processor 630 that initializes a loop based on predetermined thresholds for light levels and the slopes, derivatives, and baseline values D_{xib} stored in a memory (EEPROM) 650. Once the processor 630 has finished comparing D1 and D2 to threshold values as discussed above, it increments the loop index i in step 644 and compares the update index to the end-of-loop condition in step 446 and either exits the loop or increments the values of D1r and D2r in step 648.

If the processor 630 exits the loop, it checks the index against the total number of segments N0+N1 in the bilinear curve in step 660. If the index is less than N0+N1, the processor 630 updates the loop parameters in step 662 and restarts the loop. Otherwise, checks the ambient light level against the minimum threshold and opens or closes the aperture accordingly (steps 448, 450, and 452).

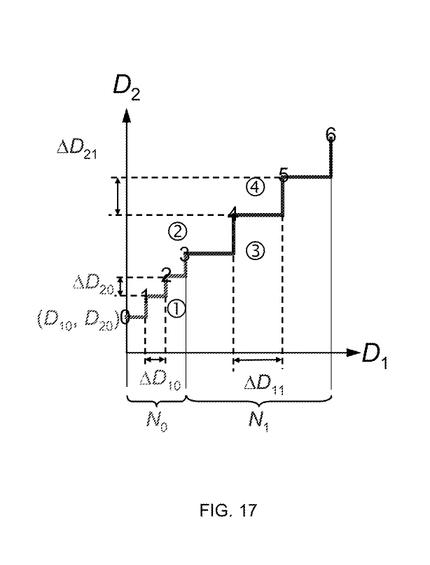

FIG. 17 illustrates a particular evolution through the loop executed by the processor 630 of FIG. 16. The sensor make four successive measurements—corresponding to points (1), (2), (3), and (4)—and the processor 630 determines whether or not those measurements correspond to points on one of the D2/D1 curves associated with an accommodative response for objects at different distances of regard (e.g., as shown in FIG. 13A) by stepping through the loop in FIG. 16. If the measurements fit to a curve, the processor 630 determines (1) that an accommodative stimulus is present and (2) the distance to the object, possibly based on which accommodative response curve the data fit. The processor 630 then opens or closes the dynamic aperture 102 as appropriate to provide the desired accommodation.

### DEFINITIONS AND CONCLUSION

As used herein, “ambient light” means light exterior to the eye. In some embodiments, ambient light refers more specifically to the light exterior to, but near or adjacent to the eye, e.g., light near the corneal surface. Ambient light can be characterized by variables such as the amount of light (e.g., intensity, radiance, luminance) and source of light (including both natural sources, e.g., sun and moon, as well as artificial sources such as incandescent, fluorescent, computer monitors, etc.).

As used herein, “accommodative response” refers to one or more physical or physiological events that enhance near vision. Natural accommodative responses, those that occur naturally in vivo, include, but are not limited to, ciliary muscle contraction, zonule movement, alteration of lens shape, iris sphincter contraction, pupil constriction, and convergence. The accommodative response can also be an artificial accommodative response, i.e., a response by an artificial optical component. Artificial accommodative responses include, but are not limited to, changing position, changing curvature, changing refractive index, or changing aperture size.

The accommodative response (also known as the accommodative loop) includes at least three involuntary ocular responses: (1) ciliary muscle contraction, (2) iris sphincter contraction (pupil constriction increases depth of focus), and (3) convergence (looking inward enables binocular fusion at the object plane for maximum binocular summation and best stereoscopic vision). Ciliary muscle contraction is related to accommodation per se: the changing optical power of the lens. Pupil constriction and convergence relate to pseudo-accommodation; they do not affect the optical power of the lens, but they nevertheless enhance near-object focusing. See, e.g., Bron A J, Vrensen G F J M, Koretz J, Maraini G, Harding 11.2000. The Aging Lens. Ophthalmologica 214:86-104.

As used herein, “accommodative impulse” refers to the intent or desire to focus on a near object. In a healthy, non-presbyopic eye, the accommodative impulse would be followed rapidly by the accommodative response. In a presbyopic eye, the accommodative impulse may be followed by a sub-optimal or absent accommodative response.

As used herein, “accommodative stimulus” is any detectable event or set of circumstances correlated to accommodative impulse or accommodative response. In the devices described herein, when an accommodative stimulus is detected by the sensor system, the sensor system preferably transmits a signal to an optical component, which in turn responds with an artificial accommodative response. Exemplary accommodative stimuli include, but are not limited to, physiological cues (such as pupil constriction and other natural accommodative responses) and environmental cues (such as ambient lighting conditions).

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more processors, perform methods that implement the various embodiments of the invention discussed above. The computer readable medium or media can be transportable, such that the program or programs stored thereon can be loaded onto one or more different processors to implement various aspects of the present invention as discussed above.

The terms “program” or “software” are used herein in a generic sense to refer to any type of computer code or set of computer-executable instructions that can be employed to program a processor to implement various aspects of embodiments as discussed above. Additionally, it should be appreciated that according to one aspect, one or more computer programs that when executed perform methods of the present invention need not reside on a single processor, but may be distributed in a modular fashion amongst a number of different processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

A hybrid flow/block diagram is used herein. The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A sensor system to detect a presence or absence of an accommodative stimulus in a human eye, the sensor system comprising:
a first sensor configured to provide a first measurement indicative of an ambient light level; and
a second sensor configured to provide a second measurement indicative of a second light level that changes in response to a physiological change triggered by the accommodative stimulus.

2. The sensor system of claim 1 wherein the first sensor includes an annular sensor.

3. The sensor system of claim 2 wherein the annular sensor has an inner diameter of about 0.9 mm to 1.2 mm and an outer diameter of about 1.1 mm to 1.3 mm.

4. The sensor system of claim 2 wherein the second sensor includes an active area disposed along a diameter of the annular sensor.

5. The sensor system of claim 4 wherein an edge of the active area and an outer diameter of the annular sensor define a gap having a length of about 250-600 μm

6. The sensor system of claim 4 wherein the active area has a width of about 30 μm to about 300 μm.

7. The sensor system of claim 4 wherein the second sensor includes another active area disposed along the diameter of the annular sensor.

8. The sensor system of claim 1 wherein at least one of the first and second sensors includes a photovoltaic cell.

9. The sensor system of claim 1 wherein the physiological change is a change in pupil diameter.

10. The sensor system of claim 1 further comprising:
a processor operably coupled to the first and second sensors and configured to determine the presence or absence of the accommodative stimulus based on the first and second measurements.

11. The sensor system of claim 10 wherein the processor is further configured to determine the presence or absence of the accommodative stimulus by comparing the first measurement and the second measurement to predetermined values representing the second measurement as a function of the first measurement in the presence of the accommodative stimulus.

12. The sensor system of claim 11 wherein the processor is further configured to compare the second measurement to the predetermined values by fitting the first and second measurements to a curve representing the predetermined values.

13. The sensor system of claim 11 further comprising:
a memory operably coupled to the processor and configured to store representations of the predetermined values.

14. The sensor system of claim 11 wherein the predetermined values are a function of age, psychological stress, and/or physiological health.

15. The sensor system of claim 10 wherein the processor is further configured to determine the presence or absence of the accommodative stimulus by (i) computing a ratio of the first measurement to the second measurement, and (ii) estimating the pupil diameter based on the ratio of the first measurement to the second measurement.

16. The sensor system of claim 10 wherein the second sensor includes a first active area and a second active area, and wherein the processor is further configured to determine the presence or absence of the physiological change by computing a difference in photocurrent generated by the first and second active areas.

17. The sensor system of claim 16 wherein the first and second active areas are disposed on opposite sides of the first sensor.

18. The sensor system of claim 10 wherein the processor is further configured to determine a status of at least one of the human eye and an environment of the human eye.

19. The sensor system of claim 10 further comprising:
a memory operably coupled to the first and second sensors and configured to store representations of the first and second measurements acquired over a predetermined interval.

20. The sensor system of claim 19 wherein the processor is further configured (i) to compute running averages of the first and second measurements acquired over the predetermined interval, and (ii) to compare the running averages to predetermined values representing the presence or absence of the accommodative stimulus.

21. The sensor system of claim 19 wherein the predetermined interval is about 0.25 seconds to about 0.50 seconds.

22. The sensor system of claim 19 wherein the memory comprises a nonvolatile storage medium.

23. The sensor system of claim 19 wherein the memory comprises an electrically erasable programmable read-only memory.

24. The sensor system of claim 10 further comprising:
an electro-active element operably coupled to the processor and configured to provide a change in effective optical power and/or depth of field in response to a signal from the processor.

25. The sensor system of claim 24 wherein the processor is further configured to place the electro-active cell in an inactive mode when at least one of the first and second measurements indicates that the ambient light level corresponds to an illuminance of less than about 5 lux.

26. The sensor system of claim 24, wherein the processor is further configured to place the electro-active cell in an active mode when at least one of the first and second measurements indicates that the ambient light level corresponds to an illuminance of greater than about 5 lux.

27. A method of distinguishing a physiological change in a human eye associated with an accommodative stimulus from other physiological changes, the method comprising:
(a) detecting an ambient light level;
(b) detecting the physiological change in the human eye; and
(c) determining a presence or absence of the accommodative stimulus based on the ambient light level and the physiological change.

28. The method of claim 27 wherein the physiological change is a change in pupil diameter.

29. The method of claim 27 wherein determining the presence of the accommodative stimulus comprises:
comparing the second measurement to predetermined values of the second measurement as a function of the first measurement in the presence of the accommodative stimulus.

30. The method of claim 29 wherein comparing the second measurement to the predetermined values comprises:
fitting the first and second measurements to a curve representing the predetermined values.

31. The method of claim 27 wherein determining the presence of the accommodative stimulus comprises:
(i) computing a ratio of the first sensor output to the second sensor output, and
(ii) estimating the pupil diameter based on the ratio of the first sensor output to the second sensor output.

32. The method of claim 27 wherein detecting the physiological change comprises detecting light on first and second sides of the pupil, and wherein determining the presence of the accommodative stimulus comprises:
computing a difference in the amount of light detected on the first and second sides of the pupil.

33. The method of claim 27 further comprising:
acquiring first and second measurements acquired over a predetermined interval;
computing running averages of the first and second measurements acquired over the predetermined interval; and
comparing the running averages to predetermined values representing the presence or absence of the accommodative stimulus.

34. The method of claim 33 wherein the predetermined interval is about 0.25 seconds to about 0.50 seconds.

35. The method of claim 33 wherein the predetermined values are a function of age, psychological stress, and/or physiological health.

36. The method of claim 27 further comprising:
changing an effective optical power and/or depth of field of the human eye based on the presence or absence of the accommodative stimulus.

37. The method of claim 36 wherein changing the effective optical power and/or depth of field includes actuating an electro-active cell.

38. The method of claim 37 further comprising:
placing the electro-active cell in an inactive mode in response to at least one of the first and second measurements indicating that the ambient light level corresponds to an illuminance of less than about 5 lux.

39. The method of claim 37 further comprising:
placing the electro-active cell in an active mode in response to at least one of the first and second measurements indicating that the ambient light level corresponds to an illuminance of greater than about 5 lux.

40. The method of claim 27 further comprising:
determining a status of at least one of the human eye and an environment of the human eye.

41. A sensor system, which, when implanted in a human eye, comprises:
(a) a first photosensor positioned at a first distance from the pupil;
(b) a second photosensor positioned a second distance from the pupil; and
(c) a processor configured to detect an accommodative stimulus based on outputs from the first and second photosensors.

42. The sensor system of claim 41 wherein the first distance is about 0.45 mm to about 0.60 mm.

43. The sensor system of claim 41 wherein the second distance is about 0.80 mm to about 1.25 mm.

44. The sensor system of claim 41, wherein the processor is further configured to detect the accommodative stimulus based on a ratio of the outputs from the first and second photosensors.

45. The sensor system of claim 41, wherein the sensor system is calibrated for an individual patient.
